(19) United States
(12) Patent Application Publication
(10) Pub. No.: US 2018/0199816 A1
(43) Pub. Date: Jul. 19, 2018

(54) URODYNAMIC ASSESSMENT SYSREMS AND METHODS

(71) Applicant: TARIS BIOMEDICAL LLC, Lexington, MA (US)

(72) Inventors: Joseph KALT, Arlington, MA (US); Dennis GIESING, Lee's Summit, MO (US); Heejin LEE, Bedford, MA (US)

(21) Appl. No.: 15/576,379

(22) PCT Filed: May 25, 2016
(86) PCT No.: PCT/US2016/034112

§ 371 Date: Nov. 22, 2017

Related U.S. Application Data
(60) Provisional application No. 62/166,404, filed on May 26, 2015.

(51) Int. Cl. A61B 5/00; A61B 5/20; A61B 5/07; A61B 5/03

(52) U.S. Cl. CPC A61B 5/0031; A61B 5/204; A61B 5/205; A61B 5/6874; A61B 2562/063; A61B 5/4845; A61B 5/036; A61B 2560/0462; A61B 2562/0247; A61B 5/07

(57) ABSTRACT

Urodynamic assessment systems, intravesical devices, and methods of their use are provided. In one embodiment, an intravesical device (200) includes an elastic body including an elongated tube defining a reservoir lumen (208), and a sensor (254) disposed at least partially in the reservoir lumen and configured to measure or detect one or more parameters. The intravesical device Cis deformable between a deployment shape for passage of the intravesical device through the urethra into the bladder and a retention shape for preventing voiding of the intravesical device through the urethra.

### CROSS-REFERENCE TO RELATED APPLICATIONS

This application claims priority to U.S. Provisional Patent Application No. 62/166,404, filed May 26, 2015, which is incorporated by reference herein.

### BACKGROUND

This disclosure is generally in the field of medical systems and methods, and more particularly in the field of urodynamic assessment systems and related methods for monitoring urodynamic measurements.

Understanding the urodynamic performance of a patient's bladder is fundamental to effective diagnosis and management of a number of urologic disorders and conditions. Urodynamic testing may be carried out when the patient experiences certain lower urinary tract symptoms, including urinary incontinence, frequent urination, painful urination, sudden, strong urges to urinate, problems starting a urine stream, problems emptying the bladder completely, and recurrent urinary tract infections. Generally, the purpose of urodynamic testing is to assess how well the bladder, sphincters, and urethra are storing and releasing urine. Although the scope of urodynamic testing varies according to the patient's overall health and specific symptoms, common tests include uroflowmetry, post-void residual measurement, cystometry, leak point pressure measurement, and pressure flow study. These tests provide objective urodynamic measurements that are useful in diagnosing various urologic conditions.

Conventional urodynamic testing is customarily performed in a physician's office and utilizes catheters for obtaining urodynamic measurements. One catheter is inserted into the patient's bladder, and another catheter is often inserted into the patient's rectum. Each catheter includes a pressure sensor, and both the bladder catheter and the rectal catheter are attached to a computerized testing system. The bladder catheter is used to completely empty the bladder and then to artificially fill the bladder with water or other stenle fluid to simulate normal filling with urine. As the bladder is being filled, the testing system monitors and records the volume of fluid instilled, bladder pressure (measured via the pressure sensor of the bladder catheter), abdominal pressure (measured via the pressure sensor of the rectal catheter), and any spasms or aberrant behaviors of the bladder wall. Meanwhile, the patient is asked to subjectively report how the bladder feels as it is being filled, including when the urge to urinate first arises and when the urge to urinate becomes overwhelming. The patient then urinates out the instilled fluid, and the testing system monitors and records the volume of fluid in the bladder, bladder pressure, and abdominal pressure throughout urination to provide a pressure trace of the void.

Although beneficial in diagnosing urologic conditions in some patients, conventional urodynamic testing presents certain drawbacks. Importantly, the testing involves artificial filling and emptying of the bladder, which is quite different from natural filling through the ureters from the kidneys and natural emptying through the urethra. Therefore, the testing fails to measure normal urodynamic performance in a natural state (i.e., throughout a natural voiding cycle). Moreover, because conventional urodynamic testing is an in-office procedure that typically is conducted over a relatively short period of less than one hour, the testing does not measure bladder behavior in a natural setting (i.e., the patient's normal environment and activity level) and presents a risk of missing any infrequent or unusual bladder events. Finally, conventional urodynamic testing requires expensive equipment, and the bladder and rectal catheters may be uncomfortable for some patients.

A need therefore exists for improved urodynamic assessment systems and methods that address one or more of the drawbacks of conventional urodynamic testing. In particular, the systems and methods should allow for measurement of normal urodynamic performance in a natural state over an extended period of time. Desirably, the urodynamic assessment systems and methods should allow for measurement of bladder behavior while the patient is in his or her normal environment and carrying out normal activities, including while the patient is awake and asleep. The systems and methods also should be well tolerated by the patient while he or she maintains a normal activity level over the testing period. The urodynamic assessment systems and methods should provide measurements over an extended period of time, improving data quality and allowing for continuous monitoring of urodynamic variables.

### BRIEF SUMMARY

Urodynamic assessment systems, intravesical devices, and methods of their use are provided. According to one aspect, an intravesical device is provided. In one embodiment, the intravesical device includes an elastic body including an elongated tube defining a reservoir lumen, and a sensor disposed at least partially in the reservoir lumen and configured to measure or detect one or more parameters. The intravesical device is deformable between a deployment shape for passage of the intravesical device through the urethra into the bladder and a retention shape for preventing voiding of the intravesical device through the urethra.

In another aspect, an urodynamic assessment system is provided. In one embodiment, the urodynamic assessment system includes an intravesical device deployable within the bladder of a patient, and an external recorder positionable outside of the body of the patient. The intravesical device includes an elastic body including an elongated tube defining a reservoir lumen, a sensor disposed at least partially in the reservoir lumen and configured to measure or detect one or more parameters, and a data transmission device disposed at least partially in the reservoir lumen and in communication with the sensor, the data transmission device configured to wirelessly transmit urodynamic measurement data. The intravesical device is deformable between a deployment shape for passage of the intravesical device through the urethra into the bladder and a retention shape for preventing voiding of the intravesical device through the urethra. The external recorder includes a data reception device configured to receive the urodynamic measurement data transmitted by the data transmission device.

In another aspect, a method for measuring urodynamic performance of the bladder of a patient is provided. In one embodiment, the method includes the step of deploying an intravesical device within the bladder. The intravesical device includes an elastic body including an elongated tube defining a reservoir lumen, and a sensor disposed at least partially in the reservoir lumen, wherein the intravesical device is deformable between a deployment shape for passage of the intravesical device through the urethra into the bladder and a retention shape for preventing voiding of the intravesical device through the urethra. The method also includes the step of measuring, via the sensor, one or more parameters.

### BRIEF DESCRIPTION OF THE DRAWINGS

FIG. 1 is an illustration of an embodiment of an urodynamic assessment system.

FIG. 2A is a plan view of an embodiment of an intravesical device, shown in a retention shape.

FIG. 2B is a plan view of the intravesical device of FIG. 2A, shown in a deployment shape.

FIG. 2C is a cross-sectional view of the intravesical device of FIG. 2A, taken along line 2C-2C.

FIG. 2D is a cross-sectional view of a portion of the intravesical device of FIG. 2A, shown in a deployment shape.

FIG. 2E is an illustration of an embodiment of an intravesical device in comparison to an approximation of the bladder trigone region.

FIG. 3 is an illustration of an embodiment of an external recorder.

FIG. 4 is an illustration of an embodiment of a gastrointestinal device.

### DETAILED DESCRIPTION

Urodynamic assessment systems and methods are provided for measuring urodynamic performance of a patient's bladder to facilitate diagnosis and management of urologic conditions. The systems and methods advantageously allow for measurement of normal urodynamic performance in a natural state over an extended period of time. In particular, the urodynamic assessment systems and methods may be used to continuously monitor urodynamic measurements as the bladder naturally fills and empties over a period of hours, days, or weeks. The systems and methods provided herein are not restricted to a physician's office; rather, they allow the patient to carry out normal activities in his or her normal environment over the assessment period. Accordingly, the urodynamic assessment systems and methods may be used to monitor bladder behavior and identify infrequent or unusual bladder events that would not be captured by conventional urodynamic testing. Instead of a bladder catheter, the systems and methods utilize an intravesical device that may be wholly deployed within the bladder and retained therein throughout the assessment period. As described below, the intravesical device can be well tolerated by the patient and, in some embodiments, may be unnoticeable to the patient. In sum, the urodynamic assessment systems and methods may provide significant improvements over conventional urodynamic testing, enhancing physicians' ability to diagnose and manage various urologic conditions.

The intravesical devices and methods of their use disclosed herein build upon the devices and methods described in U.S. Patent Application Publication No. 2009/0149833 to Cima et al., U.S. Patent Application Publication No. 2010/0331770 to Lee et al., U.S. Patent Application Publication No. 2011/0152839 to Cima et al., and U.S. Patent Application Publication No. 2012/0089121 to Lee et al., which are incorporated by reference herein.

I. Urodynamic Assessment System

Generally, the urodynamic assessment system includes an intravesical device and an external recorder. The intravesical device may be deployed within the bladder of a patient and retained therein throughout an assessment period for measuring one or more parameters. The external recorder may be disposed outside of the patient's body throughout the assessment period for receiving measurement data from the intravesical device and recording the data for subsequent analysis by a physician. In some embodiments, the urodynamic assessment system also includes a gastrointestinal device that may be deployed within the gastrointestinal tract of the patient and retained therein throughout at least a portion of the assessment period for measuring one or more parameters. The external recorder similarly may receive measurement data from the gastrointestinal device and record the data for subsequent analysis. According to various embodiments, the external recorder may receive measurement data from the intravesical device and/or the gastrointestinal device in real time, via intermittent transmission, or via a bulk download of the data (either before or after the respective device is removed from the patient's body).

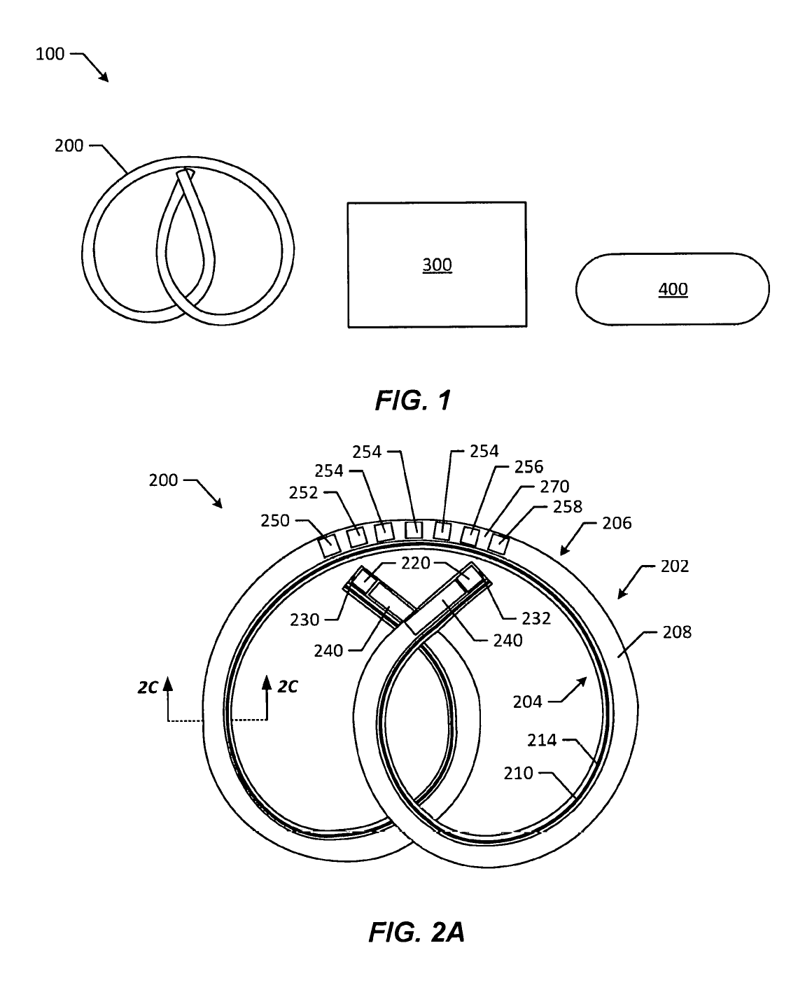

An embodiment of an urodynamic assessment system 100 is illustrated in FIG. 1. As shown, the system 100 includes an intravesical device 200 and an external recorder 300. During use of the system 100, the intravesical device 200 may be deployed within the patient's bladder and retained therein throughout an assessment period, while the external recorder 300 is disposed outside of but near the patient's body. The intravesical device 200 may be wholly implanted within the bladder, such that no portion of the device 200 extends out of the bladder. Upon deployment, the intravesical device 200 may be allowed to move freely and reorient within the bladder. As described below, the intravesical device 200 may include one or more sensors configured to measure or detect one or more parameters. The one or more parameters may be urodynamic parameters. Example parameters are bladder pressure, bladder volume, detrusor pressure, urine flow, urine density, urine composition, toxicology, and disease markers, as may be desired in certain applications. The sensors may be configured to measure or detect the desired parameters continuously or at discrete intervals throughout the assessment period. The intravesical device 200 also may include a data transmission device configured to transmit measurement data obtained via the sensors to the external recorder 300 or other device disposed outside of the patient's body. The data transmission device may be configured to transmit the measurement data continuously throughout the assessment period, intermittently at discrete intervals throughout the assessment period, or via a bulk download of the data either before or after the intravesical device 200 is removed from the patient's body.

The external recorder 300 may be in operable communication with the intravesical device 200, either continuously or at discrete intervals throughout the assessment period. As described below, the external recorder 300 may include a data reception device configured to receive the measurement data transmitted by the intravesical device 200 and a memory device configured to store the data for subsequent analysis by a physician. The external recorder 300 may be attached to the patient's body or clothing or may be otherwise worn by the patient throughout the assessment period. Alternatively, the external recorder 300 may be maintained separate from the patient's body and clothing but near the patient's body during at least a portion of the assessment period.

In some embodiments, the urodynamic assessment system 100 also includes a gastrointestinal device 400 that may be deployed within the patient's gastrointestinal tract and retained therein throughout at least a portion of the assessment period. The gastrointestinal device 400 may be swallowed by the patient or alternatively may be inserted into the patient's rectum. As described below, the gastrointestinal device 400 may include one or more sensors configured to measure or detect one or more parameters. The one or more parameters may be abdominal parameters. An example parameter is abdominal pressure, as may be desired in certain applications. The sensors may be configured to measure or detect the desired abdominal parameters continuously or at discrete intervals throughout the assessment period. The gastrointestinal device 400 also may include a data transmission device configured to transmit measurement data obtained via the sensors to the external recorder 300 or other device disposed outside of the patient's body. The data transmission device may be configured to transmit the measurement data continuously throughout the assessment period, intermittently at discrete intervals throughout the assessment period, or via a bulk download of the data either before or after the gastrointestinal device 400 is removed from the patient's body.

Although the intravesical device 200 may be used along with the external recorder 300 as a part of the urodynamic assessment system 100, the intravesical device 200 alternatively may be configured for use without the external recorder 300. For example, the intravesical device 200 may include a memory device configured to receive and store measurement data obtained via the sensors thereof throughout the assessment period, and the data may be downloaded from the memory device after removal of the intravesical device 200 from the patient. The gastrointestinal device 400 similarly may include a memory device configured to receive and store measurement data obtained via the sensors thereof throughout the assessment period, and the data may be downloaded from the memory device after removal of the gastrointestinal device 400 from the patient. According to these embodiments, the intravesical device 200 and the gastrointestinal device 400 need not include a data transmission device. Measurement data may be downloaded from the intravesical device 200 and/or the gastrointestinal device 400 onto a recorder, computer, or other device configured to receive and store the data for subsequent analysis.

The urodynamic assessment system 100 may include additional devices that are either deployed within or disposed on the patient's body for measuring additional parameters that are useful in assessing urodynamic performance. The additional devices may include one or more sensors, a data transmission device, and/or a memory device configured in a manner similar to the components of the intravesical device 200 or the gastrointestinal device 400.

Intravesical Device

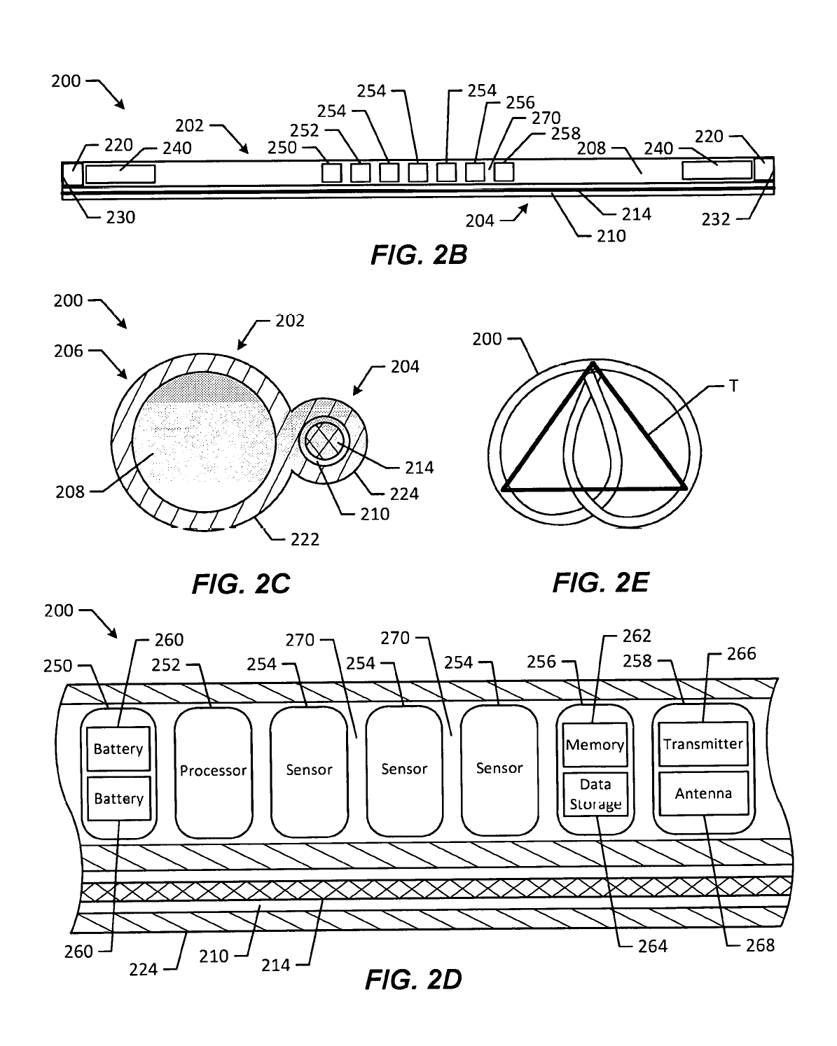

An embodiment of the intravesical device 200 is illustrated in FIGS. 2A-2E. As shown, the device 200 may include a reservoir portion 202 and a retention frame portion 204. In FIG. 2A, the device 200 is shown in a relatively expanded state suited for retention in the bladder of a patient, and in FIG. 2B, the device 200 is shown in a relatively lower-profile shape for deployment through a working channel of a deployment instrument, such as a cystoscope or other catheter. Following deployment into the bladder, the intravesical device 200 may assume the relatively expanded shape to retain the device 200 in the bladder.

For the purposes of this disclosure, terms such as “relatively expanded shape”, “relatively higher-profile shape”, or “retention shape” generally denote any shape suited for retaining the device 200 in the intended implantation location, including but not limited to the pretzel-like shape shown in FIG. 2A that is suited for retaining the intravesical device 200 in the bladder. Similarly, terms such as “relatively lower-profile shape” or “deployment shape” generally denote any shape suited for deploying the intravesical device 200 into the bladder, including the linear or elongated shape shown in FIG. 2B that is suited for deploying the device 200 through the working channel of the catheter, cystoscope, or other deployment instrument positioned in a lumen of the body, such as the urethra. In some embodiments, the intravesical device 200 may naturally assume the relatively expanded shape and may be deformed, either manually or with the aid of an external apparatus, into the relatively lower-profile shape for insertion into the bladder. Once deployed, the intravesical device 200 may spontaneously or naturally return to the initial, relatively expanded shape for retention in the bladder.

In the illustrated embodiment, the reservoir portion 202 and the retention frame portion 204 of the intravesical device 200 are longitudinally aligned and are coupled to each other along their length, although other configurations are possible. For example, the reservoir portion 202 may be attached to the retention frame portion 204 at discrete points but otherwise may be separate or spaced apart from the retention frame portion 204.

In particular, the intravesical device 200 may include an elastic or flexible device body or housing 206 that defines a reservoir lumen 208 and a retention frame lumen 210. The reservoir lumen 208 is configured to house a number of components, as described below, to form the reservoir portion 202. The retention frame lumen 210 is configured to house a retention frame 214 to form the retention frame portion 204. The illustrated lumens 208, 210 are discrete from each other, although other configurations are possible. The material used to form the device body 206 may be elastic or flexible to permit moving the intravesical device 200 between the deployment shape and the retention shape. When the device 200 is in the retention shape, the retention frame portion 204 may tend to lie inside the reservoir portion 202, as shown in FIG. 2A, although the retention frame portion 204 can be positioned inside, outside, above, or below the reservoir portion 202 in other embodiments. The flexible material also allows the device body 206 to flex outward or circumferentially expand to accommodate and/or secure components loaded into the reservoir lumen 208.

As shown in the cross-sectional view of FIG. 2C, the device body 206 includes an elongated annular tube or wall 222 that defines the reservoir lumen 208 and an elongated annular tube or wall 224 that defines the retention frame lumen 210. The tubes 222, 224 and the lumens 208, 210 can be substantially cylindrical, with the reservoir lumen 208 having a relatively larger diameter than the retention frame lumen 210, although other configurations can be selected based on, for example, the number and size of the components housed within the reservoir lumen 208, the diameter of the retention frame 214, and deployment considerations such as the inner diameter of the deployment instrument. The device body 206 may be formed integrally, such as via molding or extrusion, although separate construction and assembly of the tubes 222, 224 is possible. The tube 224 that defines the retention frame lumen 210 may extend along the entire length of the tube 222 that defines the reservoir lumen 208, so that the retention frame lumen 210 has the same length as the reservoir lumen 208 as shown, although one tube may be shorter than the other tube in other embodiments. Further, the two tubes 222, 224 are attached along the entire length of the device 200 in the illustrated embodiment, although intermittent attachment can be employed. In one example, the tube 222 of the reservoir lumen 208 has an inner diameter of about 1.5 mm and an outer diameter of about 1.9 mm, while the tube 224 of the retention frame lumen 210 has an inner diameter of about 0.5 mm and an outer diameter of about 0.9 mm. The cross-sectional area of the entire body 206 of the device 200 may be about 0.035 cm² or less.

In some embodiments, the retention frame 214 and the retention frame lumen 210 are omitted. For example, in some embodiments, the device body 206 is molded of an elastomeric material into the retention shape. In this way, the device body 206 is naturally biased to take the retention shape but can be flexed into the deployment shape.

As shown in FIG. 2A, the reservoir lumen 208 is loaded with a number of components in a serial arrangement, although other arrangements of the components are possible. The reservoir lumen 208 includes an entry 230 and an exit 232, which are shown as relatively circular openings at opposite ends of the reservoir lumen 208. The entry 230 provides ingress for the components to be placed into the reservoir lumen 208 during device loading and assembly. Once the components are loaded, at least two end plugs 220 block the entry 230 and the exit 232. The end plugs 220 may be cylindrical plugs inserted into the entry 230 and the exit 232, each having a slightly larger outer diameter than an inner diameter of the reservoir lumen 208 so that the plugs 220 substantially enclose the entry 230 and the exit 232 and are snugly retained in position. In some cases, a number of end plugs 220 can be positioned in the entry 230 or the exit 232. The end plugs 220 may be silicone plugs. The end plugs 220 also may be omitted, in which case the entry 230 and the exit 232 may be closed with a material, such as adhesive, that is placed in the reservoir lumen 208 in workable form and cures therein. Various embodiments of device bodies and end plugs are described in U.S. Patent Application Publication No. 2010/0331770 to Lee et al. The device body 206 and the end plugs 220 of the intravesical device 200 may be configured in a similar manner to provide a deformable, enclosed structure for housing components therein.

In the illustrated embodiment, the intravesical device 200 includes a pair of retention air elements 240 disposed in end portions of the reservoir lumen 208. In other embodiments, one or three or more retention air elements 240 may be included in the device 200. In other embodiments, the one or more retention air elements 240 may be located in other portions of the reservoir lumen 208 or in portions of the device 200 other than the reservoir lumen 208. The retention air elements 240 may be constructed in a number of ways to entrap a volume of air within each element 240 or within the device body 206. For example, the retention air element 240 may be a hollow capsule or a closed-cell foam, such as a foamed biocompatible polymer. The term “air” as used herein refers to any gas that is suitable for use within the body. For example, it may be actual air, carbon dioxide, nitrogen, helium, or another, preferably inert, gas. Various embodiments of retention air elements are described in U.S. Patent Application Publication No. 2012/0089121 to Lee et al. The retention air elements 240 of the intravesical device 200 may be configured in a similar manner to provide a buoyancy retention portion that may facilitate retaining the device 200 in the bladder during urination and also may enhance the device's tolerability to the patient.

In some embodiments, the retention air elements 240 are omitted. For example, if the device body 206 is formed of a relatively low density material, then the device 200 may have suitable buoyancy without the addition of one or more retention air elements.

As shown in FIGS. 2A and 2D, the intravesical device 200 includes an energy storage device 250, a processor 252, a plurality of sensors 254, a memory device 256, and a data transmission device 258 disposed in the reservoir lumen 208. Although these components are shown positioned in a central portion of the reservoir lumen 208, other positions may be used, such as toward one end of the reservoir lumen 208. Further, although the energy storage device 250, the processor 252, the sensors 254, the memory device 256, and the data transmission device 258 are shown in a serial arrangement within the reservoir lumen 208, other arrangements of these components are possible. In the illustrated embodiment, the energy storage device 250, the processor 252, the sensors 254, the memory device 256, and the data transmission device 258 are disposed entirely within the reservoir lumen 208. In other embodiments, one or more of these components may be disposed partially within the reservoir lumen 208 and partially outside of the reservoir lumen 208 or may be disposed entirely outside of the reservoir lumen 208.

The energy storage device 250 may be in communication with and configured to provide energy to or otherwise power each of the processor 252, the sensors 254, the memory device 256, and the data transmission device 258. In some embodiments, the energy storage device 250 is in communication with these components via one or more wires, although wireless configurations are possible. The energy storage device 250 may include one or more batteries 260 or other devices configured to provide energy for operation of the respective components. The one or more batteries 260 may be any suitable type of battery including, but not limited to, wet cells, dry cells, lead-acid, lithium, lithium hydride, lithium ion, or the like, at any suitable voltage and/or output current. In some embodiments, the one or more batteries 260 may be rechargeable and may be recharged by one or more other power sources. In some embodiments, the one or more batteries 260 may be wirelessly rechargeable, such as via induction or ultrasonic energy transmission.

The processor 252 may be in communication with and configured to control operation of the energy storage device 250, the sensors 254, the memory device 256, and the data transmission device 258. The processor 252 may be implemented as appropriate in hardware, software, firmware, or combinations thereof. Software or firmware implementations of the processor 252 may include computer-executable or machine-executable instructions written in any suitable programming language to perform the various functions described herein. Hardware implementations of the processor 252 may be configured to execute computer-executable or machine-executable instructions to perform the various functions described herein. The processor 252 may include, without limitation, a central processing unit (CPU), a digital signal processor (DSP), a reduced instruction set computer (RISC), a complex instruction set computer (CISC), a microprocessor, a microcontroller, a field programmable gate array (FPGA), or any combination thereof. The processor 252 also may include one or more application specific integrated circuits (ASICs) or application specific standard products (ASSPs) for handling specific data processing functions or tasks.

The sensors 254 may be configured to measure or otherwise detect one or more parameters. The one or more parameters may be urodynamic parameters. Example parameters are bladder pressure, bladder volume, detrusor pressure, urine flow, urine density, urine composition, toxicology, and disease markers. In some embodiments, each sensor 254 is configured to measure or detect a single parameter. In this manner, the plurality of sensors 254 may include a bladder pressure sensor, a bladder volume sensor, a detrusor pressure sensor, a urine flow sensor, a urine composition sensor, a toxicology sensor, and/or a disease marker sensor, or any combination thereof. Alternatively, one or more of the sensors 254 may be configured to measure or detect multiple parameters. Various types of sensors may be used to measure or detect the desired parameters, including a piezoelectric sensor, a piezoresistive sensor, a capacitive sensor, a microelectromechanical (MEMS) sensor, a fiber-optic sensor, a strain gauge, or combinations thereof. Although three sensors 254 are shown in the illustrated embodiment, the intravesical device 200 may include any number of sensors 254 for measuring any number of parameters. In some embodiments, the processor 252 is configured to direct the sensors 254 to continuously measure or detect the respective parameters throughout the assessment period. In other embodiments, the processor 252 is configured to direct the sensors 254 to intermittently measure or detect the respective parameters at discrete intervals throughout the assessment period.

In some embodiments, one or more of the sensors 254 may be isolated within the reservoir lumen 208 so that it is not in direct contact with fluids (e.g., urine) when the device 200 is deployed in the bladder. In other embodiments, all or a part of the sensor 254 may be in direct contact with fluids (e.g., urine) when the device 200 is deployed in the bladder. For example, the device body 206 may have a wall with an aperture extending therethrough. In such a case, the sensor 254 may extend out through the aperture or the sensor 254 may remain within the reservoir lumen 208 and permit fluid to enter into at least a part of the reservoir lumen 208. Other configurations are envisioned.

The memory device 256 may be in communication with each of the sensors 254 and configured to receive and store measurement data obtained via the sensors 254 throughout the assessment period. The memory device 256 may include a memory 262 and a data storage 264 in communication with one another. The memory 262 may include volatile memory (memory that maintains its state when supplied with power), such as random access memory (RAM), and/or non-volatile memory (memory that maintains its state even when not supplied with power), such as read-only memory (ROM), flash memory, ferroelectric RAM (FRAM), and so forth. The data storage 264 may include removable storage and/or non-removable storage including, but not limited to, magnetic storage, optical disk storage, and/or tape storage. The data storage 264 may provide non-volatile storage of computer-executable instructions and other data, such as the measurement data obtained via the sensors 254. The data storage 264 may store computer-executable code, instructions, or the like that may be loadable into the memory 262 and executable by the processor 252 to cause the processor 252 to perform or initiate various operations. The data storage 264 also may store data that may be copied to the memory 262 for use by the processor 252 during the execution of the computer-executable instructions. Moreover, output data generated as a result of execution of the computer-executable instructions by the processor 252 may be stored initially in the memory 262, and may ultimately be copied to the data storage 264 for non-volatile storage.

The data transmission device 258 may be in communication with the memory device 256 and configured to transmit the measurement data stored by the memory device 256 to the external recorder 300 or other device disposed outside the patient's body. The data transmission device 258 may receive the measurement data from the memory 262 or the data storage 264, depending on the configuration of the memory device 256. In some embodiments, the processor 252 is configured to direct the data transmission device 258 to continuously transmit the measurement data to the external recorder 300 throughout the assessment period. In other embodiments, the processor 252 is configured to direct the data transmission device 258 to intermittently transmit the measurement data to the external recorder 300 at discrete intervals throughout the assessment period. In still other embodiments, the processor 252 is configured to direct the data transmission device 258 to transmit the measurement data to the external recorder 300 via a bulk download of the data either before or after the intravesical device 200 is removed from the patient's body. The data transmission device 258 may include a transmitter 266 and an antenna 268 in communication with one another. The transmitter 266 and the antenna 268 may be configured to transmit communications signals, such as radio frequency (RF) signals, infrared signals, or optical signals, according to one or more established communications protocols.

In some embodiments, the components disposed in the reservoir lumen 208 may not fill the entire lumen 208. In such embodiments, a filling material may be used to fill the remainder of the reservoir lumen 208. For example, the energy storage device 250, the processor 252, the sensors 254, the memory device 256, and the data transmission device 256 may be positioned in the central portion of the reservoir lumen 208, and the filling material may be loaded in the remaining end portions of the reservoir lumen 208. The filling material may be inserted into the end portions of the reservoir lumen 208 after the lumen 208 is loaded with the other components. The filling material may be a polymeric material. The polymeric material may be placed in the reservoir lumen 208 in workable form and may cure therein. Suitable polymeric materials may cure at room temperature or in response to an external stimulus, such as heat. In some embodiments, the filling material may enclose the entry 230 and the exit 232, in which case the end plugs 220 may or may not be provided. The filling material also may be a number of end plugs 220 inserted into the end portions of the reservoir lumen 208.

Once the energy storage device 250, the processor 252, the sensors 254, the memory device 256, and the data transmission device 256 are loaded in the reservoir lumen 208, interstices or breaks 270 may be formed between adjacent components. The interstices or breaks 270 may serve as reliefs that accommodate deformation or movement of the device 200. Thus, the intravesical device 200 may be relatively flexible or deformable despite being loaded with solid components, as each component disposed within the reservoir lumen 208 may be permitted to move with respect to adjacent components. In other embodiments, the energy storage device 250, the processor 252, the sensors 254, the memory device 256, and the data transmission device 256, or some of these components, may be coupled to a flexible circuit board that is loaded in the reservoir lumen 208.

In the illustrated embodiment, the retention frame lumen 210 is loaded with the retention frame 214, which may be an elastic wire. The retention frame 214 may be configured to spontaneously return to a retention shape, such as the illustrated “pretzel” shape or another coiled shape. In particular, the retention frame 214 may retain the intravesical device 200 in the bladder. For example, the retention frame 214 may have an elastic limit and modulus that allows the device 200 to be introduced into the bladder in a relatively lower-profile shape, permits the device 200 to return to the relatively expanded shape once inside the bladder, and impedes the device from assuming the relatively lower-profile shape within the bladder in response to expected forces, such as the hydrodynamic forces associated with contraction of the detrusor muscle and urination. Thus, the intravesical device 200 may be retained in the bladder once implanted, limiting or preventing accidental expulsion. Various embodiments of retention frames are described in U.S. Patent Application Publication No. 2010/0331770 to Lee et al. The retention frame 206 of the intravesical device 200 may be configured in a similar manner to provide a deformable structure to facilitate movement of the device 200 between the deployment shape and the retention shape.

The overall configuration of the intravesical device 200 facilitates ensuring that the device 200 is tolerable to the patient. It should be noted that the device 200 may be tolerable to the patient while still being noticeable. The device 200 is both tolerable and unnoticeable in preferred embodiments, while in other embodiments the device 200 is tolerable but noticeable. A noticeable device may nonetheless be tolerable to the patient if the device is appropriately configured. For example, the intravesical device 200 may be configured to reduce the likelihood of contacting the bladder wall and to reduce the pressure exerted by the device 200 on the bladder wall when contact does occur. Bladder wall contact may cause bladder irritation that is uncomfortable for some patients and may be unbearable for sensitive patients, such as those suffering from IC/PBS. Thus, noticeability and tolerability may vary depending on differences in patient anatomy and perception of pain and discomfort. However, the overall configuration of the device 200 may ensure tolerability for most patients.

The intravesical device 200 may have a combination of characteristics that facilitates both the functionality and the tolerability of the device 200. These characteristics include the size and shape of the device 200 in combination with its compressibility and in some cases the density of the device 200, among others, which determine how mobile the device 200 is within the bladder and to what degree the device 200 contacts the trigone region or the bladder wall.

To facilitate tolerability, the intravesical device 200 may be sized so that when the device 200 is in the retention shape, the device 200 is smaller than the bladder under most conditions of bladder fullness. A device that is smaller than the bladder under most conditions of bladder fullness may have reduced contact with the bladder wall, reducing irritation of the bladder wall and contact pressure that may be sensed as bladder fullness. However, when the intravesical device 200 is in the retention shape, the device 200 may have an overall size and shape that limits the ability of the device 200 to come to rest within the bladder trigone region, which may be sensitive. FIG. 2E shows an example triangle T that approximates the trigone region of an adult human male, with the intravesical device 200 overlaying the triangle T. As shown, when the intravesical device 200 is in the retention shape, the device 200 may have an overall size and shape that is selected so that the device 200 is larger than the triangular approximation of the bladder trigone region. Such sizing also limits the likelihood of a portion of the device 200 entering or becoming trapped within the bladder neck and the ureteral orifices.

In some embodiments, the device 200 in the retention shape may have dimensions in all directions that are less than 3 cm, so that when the bladder is empty, the device 200 does not necessarily have to contact the bladder wall to fit within the bladder. In other embodiments, the device 200 in the retention shape may have at least one dimension that is larger than 3 cm. In such embodiments, the bladder wall may exert a pressure on the device 200 that compresses the device 200 in at least one direction so that it fits within the empty bladder, and the compressed device 200 may exert a return pressure on the bladder wall. The return pressure may not exceed those pressures associated with a sensation of urgency of urination or bladder fullness, so that the device 200 remains tolerable. Thus, the size and shape of the device 200 may be selected so that when the device 200 is compressed, the device 200 exerts a pressure on the bladder wall that is less than about 9.8 kPa. In some embodiments, the size and shape of the device 200 may be selected so that when the device 200 is compressed, the device 200 exerts a pressure on the bladder wall that is less than about 3.92 kPa. In particular embodiments, the size and shape of the device 200 may be selected so that when the device 200 is compressed, the device 200 exerts a pressure on the bladder wall that is less than about 1.47 kPa and may be less than 0.79 kPa. These pressures can be achieved by varying the overall size of the device 200 and the extent of its surface area. For example, the surface area of the device 200 may be increased to decrease the pressure exerted against the bladder wall upon contact, although the overall cross-sectional area of the device 200 may not be increased above a size that is deployable through the urethra.

Within the three-dimensional space occupied by the device 200 in the retention shape, the maximum dimension of the device 200 in any direction is less than 10 cm, the approximate diameter of the bladder when filled. In some embodiments, the maximum dimension of the device 200 in any direction may be less than about 9 cm, such as about 8 cm, 7 cm, 6 cm, 5 cm, 4.5 cm, 4 cm, 3.5 cm, 3 cm, 2.5 or smaller. In particular embodiments, the maximum dimension of the device 200 in any direction is less than about 7 cm, such as about 6 cm, 5 cm, 4.5 cm, 4 cm, 3.5 cm, 3 cm, 2.5 cm or smaller. In preferred embodiments, the maximum dimension of the device 200 in any direction is less than about 6 cm, such as about 5 cm, 4.5 cm, 4 cm, 3.5 cm, 3 cm, 2.5 cm or smaller.

More particularly, the three-dimensional space occupied by the device 200 is defined by three perpendicular directions. Along one of these directions the device 200 has its maximum dimension, and along the two other directions the device 200 may have smaller dimensions. For example, the smaller dimensions in the two other directions may be less than about 4 cm, such as about 3.5 cm, 3 cm. or less. In a preferred embodiment, the device 200 has a dimension in at least one of these directions that is less than 3 cm.

In some embodiments, the device 200 may have a different dimension in at least two of the three directions, and in some cases in each of the three directions, so that the device 200 is non-uniform in shape. Due to the non-uniform shape, the device 200 may be able to achieve an orientation of reduced compression in the empty bladder, which also is non-uniform in shape. In other words, there may be a particular orientation for the device 200 in the empty bladder that allows the device to exert less contact pressure against the bladder wall, making the device 200 more tolerable for the patient.

The overall shape of the intravesical device 200 may enable the device 200 to reorient itself within the bladder to reduce its engagement or contact with the bladder wall. For example, the overall exterior shape of the device 200 may be curved, and all or a majority of the exterior or exposed surfaces of the device 200 may be substantially rounded. The device 200 also may be substantially devoid of sharp edges, and is exterior surfaces may be formed from a material that experiences reduced frictional engagement with the bladder wall. Such a configuration may enable the device 200 to reposition itself within the empty bladder so that the device 200 applies lower contact pressures to the bladder wall. In other words, the device 200 may slip or roll against the bladder wall into a lower energy position, meaning a position in which the device 200 experiences less compression.

In the illustrated embodiment, the intravesical device 200 is generally planar in shape even though the device 200 occupies three-dimensional space. The device 200 may define a minor axis, about which the device 200 is substantially symmetrical, and a major axis that is substantially perpendicular to the minor axis. The device 200 may have a maximum dimension in the direction of the major axis that does not exceed about 6 cm, and in particular embodiments is less than 5 cm, such as about 4.5 cm, about 4 cm, about 3.5 cm, about 3 cm, or smaller. The device 200 may have a maximum dimension in the direction of the minor axis that does not exceed about 4.5 cm, and in particular embodiments is less than 4 cm, such as about 3.5 cm, about 3 cm, or smaller. The device 200 is curved about substantially its entire exterior perimeter in both a major cross-sectional plane and a minor cross-sectional plane. In other words, the overall exterior shape of the device 200 is curved and the cross-sectional shape of the device 200 is rounded. Thus, the device 200 is substantially devoid of edges, except for edges on the two flat ends, which are completely protected within the interior of the device 200 when the device 200 lies in a plane. These characteristics enable the device 200 to reorient itself into a position of reduced compression when in the empty bladder.

The intravesical device 200 may exhibit certain behaviors when subjected to a compression test. As described in U.S. Patent Application Publication No. 2011/0152839 to Cima et al., devices that can be compressed to a dimension of about 3 cm with an acting force of about 1 N or less were found to be tolerable within the bladder. In some embodiments, the device 200 exerts a maximum acting force of less than 1 N when the device 200 is compressed from the retention shape to a shape having a maximum dimension in any direction of about 3 cm. In other embodiments, the device 200 exerts a maximum acting force of less than about 1 N when the device 200 is compressed from the retention shape to a shape having a maximum dimension in any direction of about 1.5 cm. In some embodiments, the device 200 exerts a maximum acting force of less than about 0.5 N, less than about 0.2 N, less than about 0.1 N, or less than about 0.01 N when the device 200 is compressed from the retention shape to a shape having a maximum dimension in any direction of about 3 cm.

The intravesical device 200 also may be small enough in the retention shape to permit intravesical mobility. In particular, the device 200 when deployed may be small enough to move within the bladder, such as to move freely or unimpeded throughout the entire bladder under most conditions of bladder fullness, facilitating patient tolerance of the device 200. However, embodiments of the device 200 that otherwise move freely within the bladder may be impeded from moving freely when the bladder is completely empty, and yet the device 200 may still be tolerable if sufficiently compressible as described above.

The intravesical device 200 also may have a density that is selected to facilitate floatation. In some embodiments, the device 200 in a dry state (i.e., prior to implantation in the bladder and exposure to urine) may have a density in the range of about 0.5 g/mL to about 1.5 g/mL, such as between about 0.7 g/mL to about 1.3 g/mL. In some embodiments, the device 200 in the dry state has a density that is less than the density of water, such as a density that is less than about 1 g/mL. Such densities facilitate buoyancy and movement in the bladder. Lighter or lower density materials may be integrated into the device 200 as needed to compensate for any higher density components of the device 200, thereby maintaining an overall density that facilitates buoyancy for tolerance purposes. In addition, air or another gas may be trapped in portions of the device 200 to reduce the overall density.

U.S. Patent Application Publication No. 2011/0152839 to Cima et al. further describes various embodiments of devices having characteristics that facilitate the tolerability of the devices within the bladder. The intravesical device 200 may be configured in a similar manner to ensure patient tolerability throughout an assessment period.

External Recorder

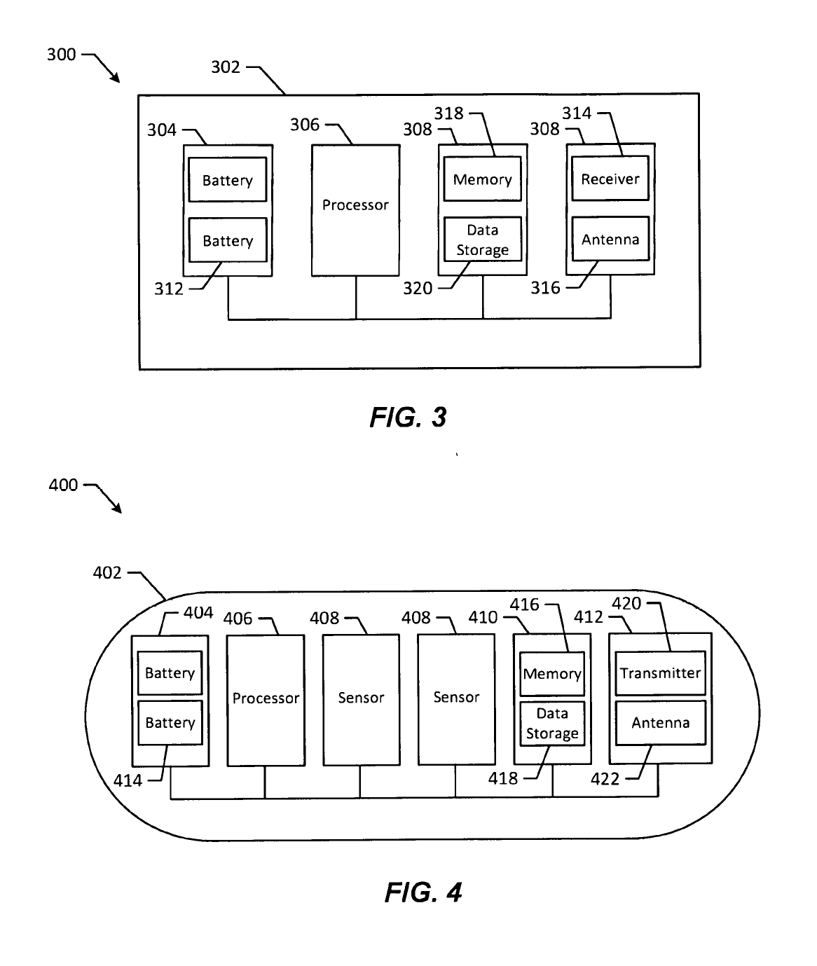

An embodiment of the external recorder 300 is illustrated in FIG. 3. During use, the external recorder 300 may be attached to the patient's body or clothing or may be otherwise worn by the patient throughout the assessment period or intermittently during portions of the assessment period. Alternatively, the external recorder 300 may be maintained separate from the patient's body and clothing but near the patient's body during at least a portion of the assessment period. As shown, the recorder 300 may include a housing 302, an energy storage device 304, a processor 306, a memory device 308, and a data reception device 310.

The energy storage device 304 may be in communication with and configured to provide energy to or otherwise power each of the processor 306, the memory device 308, and the data reception device 310. The energy storage device 304 may include one or more batteries 312 or other devices configured to provide energy for operation of the respective components. The one or more batteries 312 may be any suitable type of battery and may be rechargeable.

The processor 306 may be in communication with and configured to control operation of the energy storage device 304, the memory device 308, and the data reception device 310. The processor 306 may be implemented as appropriate in hardware, software, firmware, or combinations thereof. Software or firmware implementations of the processor 306 may include computer-executable or machine-executable instructions written in any suitable programming language to perform the various functions described herein. Hardware implementations of the processor 306 may be configured to execute computer-executable or machine-executable instructions to perform the various functions described herein.

The data reception device 310 may be configured to receive the measurement data transmitted by the intravesical device 200 as well as the measurement data transmitted by the gastrointestinal device 400. Depending upon the configuration of the intravesical device 200 and the gastrointestinal device 400, the data reception device 310 may receive the measurement data continuously throughout the assessment period, intermittently at discrete intervals throughout the assessment period, or via a bulk download of the data before or after the respective device is removed from the patient's body. The data reception device 310 may include a receiver 314 and an antenna 316 in communication with one another. The receiver 314 and the antenna 316 may be configured to receive communications signals, such as radio frequency (RF) signals, infrared signals, or optical signals, according to one or more established communications protocols.

The memory device 308 may be in communication with the data reception device 310 and configured to receive and store the measurement data received by the data reception device 310 throughout the assessment period. The memory device 308 may include a memory 318 and a data storage 320 in communication with one another. The memory 318 may include volatile memory and/or non-volatile memory. The data storage 320 may include removable storage and/or non-removable storage.

Gastrointestinal Device

An embodiment of the gastrointestinal device 400 is illustrated in FIG. 4. During use, the gastrointestinal device 400 may be deployed within the patient's gastrointestinal tract and retained therein throughout at least a portion of the assessment period. The gastrointestinal device 400 may be swallowed by the patient or alternatively may be inserted into the patient's rectum. As shown, the gastrointestinal device 400 may include a housing 402, an energy storage device 404, a processor 406, a plurality of sensors 408, a memory device 410, and a data transmission device 412.

The energy storage device 404 may be in communication with and configured to provide energy to or otherwise power each of the processor 406, the sensors 408, the memory device 410, and the data transmission device 412. The energy storage device 404 may include one or more batteries 414 or other devices configured to provide energy for operation of the respective components. The one or more batteries 414 may be any suitable type of battery and may be rechargeable.

The processor 406 may be in communication with and configured to control operation of the energy storage device 404, the sensors 408, the memory device 410, and the data transmission device 412. The processor 406 may be implemented as appropriate in hardware, software, firmware, or combinations thereof. Software or firmware implementations of the processor 406 may include computer-executable or machine-executable instructions written in any suitable programming language to perform the various functions described herein. Hardware implementations of the processor 406 may be configured to execute computer-executable or machine-executable instructions to perform the various functions described herein.

The sensors 408 may be configured to measure or otherwise detect one or more parameters. The one or more parameters may be abdominal parameters. An example parameter is abdominal pressure. In some embodiments, each sensor 408 is configured to measure or detect a single parameter. Alternatively, one or more of the sensors 408 may be configured to measure or detect multiple parameters. Although two sensors 408 are shown in the illustrated embodiment, the gastrointestinal device 400 may include any number of sensors 408 for measuring any number of parameters. In some embodiments, the processor 406 is configured to direct the sensors 408 to continuously measure or detect the respective parameters throughout the assessment period. In other embodiments, the processor 406 is configured to direct the sensors 408 to intermittently measure or detect the respective parameters at discrete intervals throughout the assessment period.

The memory device 410 may be in communication with each of the sensors 408 and configured to receive and store measurement data obtained via the sensors 408 throughout the assessment period. The memory device 410 may include a memory 416 and a data storage 418 in communication with one another. The memory 416 may include volatile memory and/or non-volatile memory. The data storage 418 may include removable storage and/or non-removable slot age.

The data transmission device 412 may be in communication with the memory device 410 and configured to transmit the measurement data stored by the memory device 410 to the external recorder 300 or other device disposed outside the patient's body. The data transmission device 410 may receive the measurement data from the memory 416 or the data storage 418, depending on the configuration of the memory device 410. In some embodiments, the processor 406 is configured to direct the data transmission device 412 to continuously transmit the measurement data to the external recorder 300 throughout the assessment period. In other embodiments, the processor 406 is configured to direct the data transmission device 412 to intermittently transmit the measurement data to the external recorder 300 at discrete intervals throughout the assessment period. In still other embodiments, the processor 406 is configured to direct the data transmission device 412 to transmit the measurement data to the external recorder 300 via a bulk download of the data either before or after the gastrointestinal device 400 is removed from the patient's body. The data transmission device 412 may include a transmitter 420 and an antenna 422 in communication with one another. The transmitter 420 and the antenna 422 may be configured to transmit communications signals, such as radio frequency (RF) signals, infrared signals, or optical signals, according to one or more established communications protocols.

II. Use and Applications of the Urodynamic Assessment System

The urodynamic assessment system 100 may be used for measuring urodynamic performance of a patient's bladder to facilitate diagnosis and management of urologic conditions. In particular, the system 100 may be used to continuously monitor urodynamic measurements as the bladder naturally fills and empties over an extended period of time, such as multiple hours, days, or weeks. The system 100 may allow the patient to carry out normal activities in his or her normal environment over the assessment period; while measurement data is collected. The system 100 may be used with humans, whether male or female, adult or child, or in other mammals, such as for veterinary or livestock applications.

In one example, the intravesical device 200 is used in combination with the external recorder 300, such that urodynamic measurement data is obtained by the device 200 and stored by the recorder 300 throughout the assessment period. The intravesical device 200 may be implanted by passing the device 200 through a deployment instrument into the patient's bladder. The deployment instrument may be any suitable lumen device, such as a catheter, urethral catheter, or cystoscope. The intravesical device 200 may assume a retention shape, such as the pretzel-like shape shown in FIG. 2A, once the device 200 emerges from the deployment instrument into the bladder. The external recorder 300 may be disposed outside of but near the patient's body throughout the assessment period. In particular, the recorder 300 may be attached to the patient's body or clothing or may be otherwise worn by the patient.

Upon deployment of the intravesical device 200 within the bladder, the device 200 may measure or detect one or more parameters via the one or more sensors 254 thereof. In particular, the one or more sensors 254 may measure or detect one or more urodynamic parameters. The one or more parameters may include bladder pressure, bladder volume, detrusor pressure, urine flow, urine composition, toxicology, or disease markers, or any combination thereof. In some embodiments, the sensors 254 continuously measure or detect the respective parameters throughout the assessment period. In other embodiments, the sensors 254 intermittently measure or detect the respective parameters at discrete intervals throughout the assessment period. Based upon the measurements obtained by the sensors 254, the processor 252 may generate measurement data that is directed to and stored by the memory device 256 of the intravesical device 200. In particular, the measurement data may be stored by the memory 262 and/or the data storage 264 of the memory device 256. The measurement data may include a timestamp for each measurement obtained, allowing for later correlation of the measurements over time.

The intravesical device 200 may wirelessly transmit the measurement data to the external recorder 300 throughout the assessment period. In particular, the data reception device 258 of the intravesical device 200 may wirelessly transmit the measurement data to the data reception device 310 of the external recorder 300. In some embodiments, the intravesical device 200 continuously transmits the measurement data to the external recorder 300 throughout the assessment period. In other embodiments, the intravesical device 200 intermittently transmits the measurement data at discrete intervals throughout the assessment period. Upon receiving the measurement data from the intravesical device 200, the external recorder 300 may store the data via the memory device 308 thereof.

Throughout the assessment period, the intravesical device 200 may continue to measure or detect the one or more parameters and transmit the measurement data to the external recorder 300. According to various embodiments, the assessment period may be 12 hours, 24 hours, 48 hours, 7 days, 14 days, 21 days, 28 days, or more. At the end of the assessment period, the intravesical device 200 may be removed from the bladder, such as with a retrieval instrument, and the external recorder 300 may be removed from the patient's body. The measurement data stored by the external recorder 300 may be downloaded onto a computer or other device and analyzed by a physician to diagnose or manage various urologic conditions.

In another example, the intravesical device 200 is used without the external recorder 300, such that urodynamic measurement data is obtained by and stored by the device 200 throughout the assessment period. The intravesical device 200 may be implanted within the bladder and may measure or detect one or more parameters via the one or more sensors 254 in a manner similar to that described above. Based upon the measurements obtained by the sensors 254, the processor 252 may generate measurement data that is directed to and stored by the memory 262 and/or the data storage 264 of the memory device 256. The measurement data may include a timestamp for each measurement obtained, allowing for later correlation of the measurements over time. All of the measurement data obtained by the intravesical device 200 throughout the assessment period may be stored by the memory device 256, instead of being transmitted outside of the patient's body. Accordingly, the intravesical device 200 need not include a data transmission device 258. At the end of the assessment period, the intravesical device 200 may be removed from the bladder, and the measurement data stored by the device 200 may be downloaded onto a computer or other device for subsequent analysis.

In either of the above examples, the gastrointestinal device 400 also may be used, in addition to the intravesical device 200, to obtain abdominal measurement data that may be useful in assessing urodynamic performance. The gastrointestinal device 400 may be deployed within the patient's gastrointestinal tract and retained therein throughout at least a portion of the assessment period. In some embodiments, the gastrointestinal device 400 is swallowed by the patient. In other embodiments, the gastrointestinal device 400 is inserted into the patient's rectum.

Upon deployment of the gastrointestinal device 400, the device 400 may measure or detect one or more parameters via the one or more sensors 408 thereof. In particular, the one or more sensors 408 may measure or detect one or more abdominal parameters. The one or more parameters may include abdominal pressure. In some embodiments, the sensors 408 continuously measure or detect the respective parameters throughout the assessment period or a portion thereof. In other embodiments, the sensors 408 intermittently measure or detect the respective parameters at discrete intervals throughout the assessment period or a portion thereof. Based upon the measurements obtained by the sensors 408, the processor 406 may generate measurement data that is directed to and stored by the memory device 410 of the gastrointestinal device 400. In particular, the measurement data may be stored by the memory 416 and/or the data storage 418 of the memory device 410. The measurement data may include a timestamp for each measurement obtained, allowing for later correlation of the measurements over time. In embodiments in which the external recorder 300 is used, the gastrointestinal device 400 may wirelessly transmit the measurement data to the external recorder 300 throughout the assessment period, either continuously or intermittently at discrete intervals, in a manner similar to the intravesical device 200. In embodiments in which the external recorder 300 is not used, all of the measurement data obtained by the gastrointestinal device 400 throughout the assessment period may be stored by the memory device 410, in a manner similar to the intravesical device 200.

Publications cited herein and the materials for which they are cited are specifically incorporated by reference. Modifications and variations of the systems, devices, and methods described herein will be obvious to those skilled in the art from the foregoing detailed description. Such modifications and variations are intended to come within the scope of the appended claims.

1. An intravesical device deployable within the bladder of a patient, the intravesical device comprising:
an elastic body comprising an elongated tube defining a reservoir lumen; and
a sensor disposed at least partially in the reservoir lumen and configured to measure or detect one or more parameters,
wherein the intravesical device is deformable between a deployment shape for passage of the intravesical device through the urethra into the bladder and a retention shape for preventing voiding of the intravesical device through the urethra.

2. The intravesical device of claim 1, wherein the one or more parameters comprise bladder pressure, bladder volume, detrusor pressure, urine flow, urine density, urine composition, toxicology, or disease markers.

3. The intravesical device of claim 1, wherein the sensor is disposed in a central portion of the reservoir lumen.

4. The intravesical device of claim 1, wherein the intravesical device comprises a plurality of sensors disposed at least partially in the reservoir lumen and configured to detect one or more parameters.

5. The intravesical device of claim 1, further comprising an energy storage device disposed at least partially in the reservoir lumen and in communication with the sensor.

6. (canceled)

7. The intravesical device of claim 1, further comprising a processor disposed at least partially in the reservoir lumen and in communication with the sensor.

8. The intravesical device of claim 1, further comprising a memory device disposed at least partially in the reservoir lumen and in communication with the sensor.

9. The intravesical device of claim 8, wherein the memory device comprises a memory and a data storage.

10. The intravesical device of claim 1, further comprising a data transmission device disposed at least partially in the reservoir lumen and in communication with the sensor.

11. The intravesical device of claim 10, wherein the data transmission device comprises a transmitter and an antenna.

12. The intravesical device of claim 1, wherein the retention shape has a maximum dimension in any dimension of 6 cm or less when in an uncompressed state, and wherein the intravesical device exerts a maximum acting force less than 1 N when the intravesical device is compressed from the retention shape to a shape having a maximum dimension in any dimension of 3 cm.

13. The intravesical device of claim 12, wherein the intravesical device exerts a maximum acting force less than 1 N when the intravesical device is compressed from the retention shape to a shape having a maximum dimension in any dimension of 1.5 cm.

14. (canceled)

15. The intravesical device of claim 1, wherein the intravesical device in a dry state has a density from 0.5 g/mL to 1.5 g/mL.

16. The intravesical device of claim 1, wherein the retention shape has two sub-circles, each having its own smaller arches and sharing a common larger arch.

17. The intravesical device of claim 1, further comprising a retention frame lumen and a retention frame disposed in the retention frame lumen.

18. (canceled)

19. The intravesical device of claim 1, further comprising a retention air element comprising a volume of entrapped air.

20. The intravesical device of claim 19, wherein the retention air element is disposed in the reservoir lumen.

21. A urodynamic assessment system for measuring urodynamic performance of the bladder of a patient, the system comprising:
an intravesical device deployable within the bladder, the intravesical device comprising:
an elastic body comprising an elongated tube defining a reservoir lumen;
a sensor disposed at least partially in the reservoir lumen and configured to measure or detect one or more parameters; and
a data transmission device disposed at least partially in the reservoir lumen and in communication with the sensor, the data transmission device configured to wirelessly transmit urodynamic measurement data,
wherein the intravesical device is deformable between a deployment shape for passage of the intravesical device through the urethra into the bladder and a retention shape for preventing voiding of the intravesical device through the urethra; and
an external recorder positionable outside of the body of the patient, the external recorder comprising a data reception device configured to receive the urodynamic measurement data transmitted by the data transmission device.

22. The system of claim 21, wherein the one or more parameters comprise bladder pressure, bladder volume, detrusor pressure, urine flow, urine density, urine composition, toxicology, or disease markers.

23. The system of claim 21, wherein the external recorder further comprises a memory device in communication with the data reception device and configured to store the urodynamic measurement data.

24. The system of claim 21, further comprising a gastrointestinal device deployable within the gastrointestinal tract of the patient, the gastrointestinal device comprising:
a housing;
a sensor disposed in the housing and configured to measure or detect abdominal pressure and/or one or more other abdominal parameters; and
a data transmission device disposed in the housing and in communication with the sensor, the data transmission device configured to wirelessly transmit abdominal measurement data to the data reception device of the external recorder.

25. (canceled)

26. A method for measuring urodynamic performance of the bladder of a patient, the method comprising:
deploying the intravesical device of claim 1 within the bladder; and
measuring, via the sensor, one or more parameters.

27. The method of claim 26, wherein the one or more parameters comprise bladder pressure, bladder volume, detrusor pressure, urine flow, urine density, urine composition, toxicology, or disease markers.

28. The method of claim 26, further comprising:
positioning an external recorder outside of the body of the patient; and
wirelessly transmitting urodynamic measurement data from the intravesical device to the external recorder.

29. The method of claim 28, further comprising:
deploying a gastrointestinal device within the gastrointestinal tract of the patient, the gastrointestinal device comprising:
a housing; and
a sensor disposed in the housing; and
measuring, via the sensor, abdominal pressure and/or one or more other abdominal parameters.

30. (canceled)

31. The method of claim 29, further comprising wirelessly transmitting abdominal measurement data from the gastrointestinal device to the external recorder.
